(19) United States
(12) Patent Application Publication
(10) Pub. No.: US 2022/0054651 A1
(43) Pub. Date: Feb. 24, 2022

(54) TERNARY MICELLAR COMPLEX COMPOSED OF A SIALOGLYCOSPHINGOLIPID, A THERAPEUTICALLY ACTIVE SUBSTANCE AND AN ANTIBODY

(71) Applicants: CONSEJO NACIONAL DE INVESTIGACIONES CIENTÍFICAS Y TÉCNICAS (CONICET), CABA (AR); CENTRO DE EXCELENCIA EN PRODUCTOS Y PROCESOS (CEPRECOR), Provincia de Córdoba (AR)

(72) Inventors: Dante Miguel BELTRAMO, Ciudad de Cordoba (AR); Ariel Gustavo GARRO, Ciudad de Cordoba (AR); Roxana Valeria ALASINO, Ciudad de Cordoba (AR); Victoria LEONHARD, Ciudad de Cordoba (AR)

(73) Assignees: Consejo Nacional de Investigaciones Científicas y Técnicas (CONICET), Caba (AR); Centro de Excelencia en Productos y Procesos (CEPRECOR), Provincia de Cordoba (AR)

(21) Appl. No.: 17/413,923

(22) PCT Filed: Dec. 14, 2018
(86) PCT No.: PCT/PE2018/000033

§ 371 Date: Jun. 14, 2021

(51) Int. Cl. A61K 47/69; A61K 31/337; A61K 31/704; A61K 31/7048; A61K 9/107; A61K 9/19; A61K 47/68

(52) U.S. Cl. CPC A61K 47/6907; A61K 31/337; A61K 31/704; A61K 47/6803; A61K 9/1075; A61K 9/19; A61K 31/7048

(57) ABSTRACT

The invention relates to a pharmaceutical composition soluble in an aqueous solution, which is characterised in that it comprises sialoglycosphingolipids; polypeptides selected from the group comprising antibodies, fragments and variants of same; and at least one therapeutically active substance, forming a ternary micellar complex of sialoglycosphingolipid-therapeutically active substance-polypeptide in which the polypeptides are non-covalently associated with the micelles formed by the sialoglycosphingolipid and the therapeutically active substance.

### FIELD OF INVENTION

The present invention belongs to the field of water-soluble pharmaceutical compounds for the administration of therapeutically active substances, wherein said compounds are micelles coated with polypeptides which allows the efficient administration of the therapeutically active substance to a certain desired site of action.

### STATE OF THE ART

One of the critical points of chemotherapy and, particularly oncological chemotherapy, is the arrival at the site of action. In this regard, innumerable alternatives have been tested to achieve specific targeting. For example, liposomes can be targeted to the lung when coated with 0-stearoyl amylopectin and polyoxyethylene or monosialogangliosides (Deol P, Khuller G K. (1997) “Lung specific stealth liposomes: stability, biodistribution and toxicity of liposomal antitubercular drugs.” Biochim Biophys Acta vol. 1334:161-72). Also, with this same objective, several molecules have been incorporated on pegylated liposomes to give them directionality, among which can be mentioned folic acid, thiamine, peptides such as Arginine-Glycine-Aspartic Acid, sugars such as galactose, antibodies and antibody fragments such as anti-HER2, nucleic acid aptamers and proteins such as transferrin. These molecules allow the liposome to target tumor tissue and not healthy cells. Among these molecules, antibodies stand out, especially monoclonal antibodies that have been used for the treatment of tumors in the colon, ovary, prostate, etc. Recently it has been demonstrated that immunoliposomes with anti-GD2 antibodies, containing an antitumor drug like fenretinide, which induces apoptosis in neuroblastoma and melanoma cell lines, possess strong anti-neuroblastoma activity both in vitro and in vivo (Raffaghello L, Pagnan G, Pastorino F, et al. Immunoliposomal fenretinide: a novel antitumor drug for human neuroblastoma. Cancer Lett. 2003; 197:151-155). The Fab fragment of IgG has also been used in immunoliposomes with disialogangliosides to inhibit neuroblastoma growth and metastasis in animal models. Moreover, Doxorubicin (Doxil) liposomes loaded with anti-HER2 monoclonal antibodies exhibit greater cytotoxic effect than those liposomes without the antibodies (Park J W, Kirpotin D B, Hong K, et al. Tumor targeting using anti-her2 immunoliposomes. J Control Release. 2001; 74:95-113) as they produce greater inhibition of tumors overexpressing this receptor.

Another possibility for drug delivery is the use of polymer nanoparticles as drug transporters, which have the advantage that they accumulate in the tumor area due to the increased permeability caused by defects in the vasculature. Another advantage is that drug transport by these nanoparticles often includes a sustained release system that improves bioavailability and reduces systemic effects (Krasnici, S., Werner, A., Eichhorn, M. E., Schmitt-Sody, M., Pahernik, S. A., Sauer, B., Schulze, B., Teifel, M., Michaelis, U., Naujoks, K., and Dellian, M., Effect of the Surface Charge of Liposomes on their Uptake by Angiogenic Tumor Vessels, International Journal of Cancer 105 (4), 561-567, 2003, —Lucarini, M., Franchi, P., Pedulli, G. F., Pengo, P., Scrimin, P., and Pasquato, L, EPR study of dialkyl nitroxides as probes to investigate the exchange of solutes between the ligand shell of monolayers of protected gold nanoparticles and aqueous solutions, Journal of the American Chemical Society 126 (30), 9326-9329, 2004). However, they have the great disadvantage of these nanoparticles is that they have a low selectivity towards cancer cells so there is still a need to develop site-specific therapies.

The monoclonal antibody 2C5 is able to bind to various types of cancer cells, by interacting with the cell surface to bind to nucleosomes released from neighboring cells that enter death by apoptosis. The 2C5-Doxil complex, being Doxil, Doxorubicin-loaded liposomes, recognizes and destroys various types of cancer cells such as lung, breast and colon cancer cells more effectively than Doxil alone (Lukyanov A N, Elbayoumi T A, Chakilam A R, Torchilin V P. Tumor-targeted liposomes: doxorubicin-loaded long-circulating liposomes modified with anti-cancer antibody. J Control Release. 2004; 100:135-144).

Selective or site-specific therapy is that in which the drugs used act specifically on the target tissue, organ or cell. This type of site-specific therapy allows the treatment to be more effective and reduces unwanted non-specific side effects. While several strategies have been developed to achieve this goal, there is still a need to develop formulations that allow site-specific therapy for the treatment of cancer, especially in the case of solid tumors, for which drugs have more impediments in reaching the specific destination (Poste, G. and Kirsh, R., Site-specific (targeted) drug delivery in cancer-therapy, Bio-Technology 1 (10), 869-878, 1983, —Brigger, I., Dubernet, C., and Couvreur, P., Nanoparticles in cancer therapy and diagnosis, Advanced Drug Delivery Reviews 54 (5), 631-651, 2002).

To achieve this type of therapy, the production of over-expression of antigens associated with the different tumors or of receptors that selectively capture specific molecules related to tumor growth, is very advantageous. It allows the targeting of the drugs by means of nanoparticles with specific ligands, which recognize these over-expressed antigens or receptors in tumor cells. In the case of antibodies, the two properties of the immunoglobulin are often combined: specific recognition and biological activity per se, as is the case, for example, with Trastuzumab (Herceptin®). This is a humanized monoclonal antibody that specifically binds to the HER2/neu membrane region with very high affinity, inhibiting both signal translation mechanisms and cell proliferation, which appears to be an excellent strategy for targeting drugs due to the accessibility of HER2. Trastuzumab, was the first targeted therapy approved by the FDA for the treatment of metastatic breast cancer, as first-line treatment in combination with Paclitaxel (Ptx), or in monotherapy for patients who have received at least one prior chemotherapy treatment (Vogel, C. L., Cobleigh, M. A., Tripathy, D., Gutheil, J. C., Harris, L. N., Fehrenbacher, L., Slamon, D. J., Murphy, M., Novotny, W. F., Burchmore, M., Shak, S., Stewart, S. J., and Press, M. Efficacy and safety of trastuzumab as a single agent in first-line treatment of HER2-overexpressing metastatic breast cancer, Journal of Clinical Oncology 20 (3), 719-726, 2002).

On the other hand, synergistic antitumor effects have also been described when Trastuzumab is administered in combination with other chemotherapeutic agents such as Paclitaxel and Doxorubicin, with reports of favorable response of up to 73% in three phase II trials. (Burstein, H. J., Harris, L. N., Gelman, R., Lester, S. C., Nunes, R. A., Kaelin, C. M., Parker, L. M., Ellisen, L. W., Kuter, I., Gadd, M. A., Christian, R. L, Kennedy, P. R., Borges, V. F., Bunnell, C. A., Younger, J., Smith, B. L., and Winer, E. P., Preoperative therapy with trastuzumab and paclitaxel followed by sequential adjuvant doxorubicin/cyclophosphamide for HER2 overexpressing stage II or Ill breast cancer: A pilot study, Journal of Clinical Oncology 21 (1), 46-53, 2003—Slamon, D. J., Leyland-Jones, B., Shak, S., Fuchs, H., Pat6n, V., Bajamonde, A., Fleming, T., Eiermann, W., Wolter, J., Pegram, M., Baselga, J., and Norton, L., Use of chemotherapy plus a monoclonal antibody against HER2 for metastatic breast cancer that overexpresses HER2, New England Journal of Medicine 344 (11), 783-792, 2001; Coudert, B. P., Arnould, L, Moreau, L., Chollet, P., Weber, B., Vanlemmens, L, Molucon, C., Tubiana, N., Causeret, S., Misset, J. L, Feutray, S., Mery-Mignard, D., Garnier, J., and Fumoleau, P., Pre-operative systemic (neo-adjuvant) therapy with trastuzumab and docetaxel for HER2-overexpressing stage II or Ill breast cancer: results of a multicenter phase 11 trial, Annals of Oncology 17 (3), 409-414, 2006).

The incorporation of specific monoclonal antibodies against a given antigen on the surface of nanocarriers could achieve a doble effect, on one hand, that of the antibody itself and on the other, the effect of the drugs present in the nanostructure.

While nanocarriers, in the form of liposomes or micelles, which can be associated with antibodies to achieve targeted delivery of active ingredients, have been described in the state of the art, such as Ahn J et al, Antibody fragment-conjugated polymeric micelles incorporating platinum drugs for targeted therapy of pancreatic cancer; Biomaterials. 2015 January; 39:23-30, or Vladimir P. Torchilin et al, Immunomicelles: Targeted pharmaceutical carriers for poorly soluble drugs. Proc Natl Acad Sel USA. 2003 May 13; 100(10): 6039-6044.). While these papers teach strategies for modifying liposomes or micellar structures to be able to achieve direct delivery of a drug, the strategies involve functionalization of such carriers in order to achieve an association between the surface of the carrier and the antibody. As an example, U.S. Pat. No. 6,214,388 (B1), from the University of California, describes immunoliposomes comprising liposomal structures that have antibodies on their surface that are bound to lipids derivatized with Polyethylene glycol.

Other examples of ways to associate an antibody to the surface of a nanoparticle, may include: Conjugación de Trastuzumab, sobre nanoparticulas: Bingfeng Sun 1, Heni Rachmawati 2, Yutao Liu 3, Jing Zhao 1, Si-Shen Feng en el capiulo “Antibody-Conjugated Nanoparticles of Biodegradable Polymers for Targeted Drug Delivery” del libro Bionanotechnology II: Global Prospects by E. Reisner David, August 2010. Another example, Anti-CD8 with a nanoparticles de polyglycolic: Anti-CD8 conjugated nanoparticles to target mammalian cells expressing CD8—A. Bicho, Inés N. Pee, a, A. C. A. Roque, M. Margarida Cardoso*—International Journal of Pharmaceutics 399 (2010) 80-86. Another example is Cetuximab bound to albumin nanoparticles. Cetuximab (Erbitux) is a humanized monoclonal antibody directed against the epidermal growth factor receptor (EGFR) that was approved by the United States Food and Drug Administration (FDA) in 2004 for the treatment of colorectal cancer. Clin Ther 2005; 27: 684-94). More recently Karin L. et al have developed human serum albumin (HSA) nanoparticles coupled to monoclonal antibodies such as cetuximab. The particles obtained in this case show a size ranging between 200 and 250 nanometers. (Targeted human serum albumin nanoparticles for specific uptake in EGFR-Expressing colon carcinoma cells, —Karin Low, Matthias Wacker, Sylvia Wagner, Klaus Langer, Hagen von Briesen, Nanomedicine: Nanotechnology, Biology, and Medicine (2011)) (Targeted human serum albumin nanoparticles for specific uptake in EGFR-Expressing colon carcinoma cells, Karin Löw, Dipl.—Biol.a, Matthias Wacker, PhDb, Sylvia Wagner, PhDa, Klaus Langer, PhDc, Hagen von Briesen, PhDa, Nanomedicine: Nanotechnology, Biology, and Medicine (2011).

As can be seen in the above examples mention above, both the carriers used (liposomes or micelles functionalized with antibodies) as well as the procedures used to incorporate the antibodies, they all present several different steps to reach the Ac-nanoparticle or Antibody-liposome or antibody-micelle complex, but the most important of these procedures is that, in all cases the use of different chemical crosslinking agents to covalently bind or chemically conjugate the antibody to different parts of the nanoparticles used is mentioned.

The use of these crosslinking agents is an undesirable condition in this type of coupling, since it is well known that these reagents can bind to specific groups such as aminos (NH2) or carboxyls (COOH) in different places of therapeutically active proteins or nanoparticles, and from here bind to antibodies and can produce changes in the biological activity of antibodies (decrease in activity) as well as in other proteins with biological activity such as enzymes.

Although attempts have been made to incorporate on the surface of micellar structures, loaded with therapeutically active substances, antibodies or fragments or variants thereof that allow the targeting of a particular tissue or diseased organ, functionalization processes of the structural compounds of the micelles or the use of linkers that make the antibody/micelle interaction efficient are still required.

The novelty and relevance of the incorporation of the antibodies on the ganglioside micelles described in this invention lies in the fact that it is the first drug nanocarrier that can be coated non-covalently, without the need to derivatize or modify the micelle components to achieve the interaction between the micelle and the antigen recognition polypeptide, such as antibodies.

### BRIEF DESCRIPTION OF THE INVENTION

The pharmaceutical composition soluble in aqueous solution, object of the present invention, comprises sialoglycosphingolipids; polypeptides selected from the set comprising antibodies, fragments and variants thereof; at least one therapeutically active substance; forming a ternary micellar complex, sialoglycosphingolipid-therapeutically active substance-Polypeptide, wherein said polypeptides are non-covalently associated with the micelles formed by said sialoglycosphingolipids and said therapeutically active substance. Where preferably, said polypeptides coat said micelles. Wherein said sialoglycosphingolipids are selected from the set comprising monosialogangliosides, disialogangliosides, trisialoganglioside, GM1, LIGA-GM1, GM2, GD1a, GD1b, GT1 and mixtures thereof.

Wherein said polypeptides are selected from the set comprising human polyclonal antibodies, humanized polyclonal antibodies, humanized monoclonal antibodies, polyclonal antibodies of animal origin, monoclonal antibodies of animal origin, chimeric antibodies, fragments thereof and mixtures thereof. Wherein said therapeutically active substance is selected from the set comprising antitumor drugs, antifungal drugs, hydrophobic drugs, hydrophilic drugs, taxanes, paclitaxel, docetaxel, doxorubicin, amphotericin, prostaglandins, isosorbide dinitrite, prostaglandins, propofol, isosorbide dinitrate, testosterone, nitroglycerin, estradiol, vitamin E, cortisone, dexamethasone and its esters and betamethasone valerate, preferably paclitaxel, docetaxel, doxorubicin and amphotericin; in a molar ratio of therapeutically active substance/sialoglycosphingolipids ranging from 1:2 to 1:100. And comprising a molar ratio of therapeutically active substance/sialoglycosphingolipids ranging from 1:2 to 1:100.

Preferably the composition of the invention is sterile injectable and translucent, wherein said micellar complex possesses an average size less than 100 nm, more preferably an average size comprised between 10 nm and 60 nm. Furthermore, the pharmaceutical composition of the invention directs the administration of the therapeutically active substance towards the target of said antibody. Furthermore, the pharmaceutical composition of the invention comprises said micellar complex in the absence of albumin, linkers, crosslinking agents and derivatized molecules.

Furthermore, in a preferred version of the formulation of the present invention, said polypeptides, which are antibodies, fragments or variants of antibodies, when they already form part of the ternary micellar complex, retain the ability to bind to their ligand. That is to say that they maintain their natural biological activity.

Another object of the present invention is a pharmaceutical composition, soluble in aqueous solution comprising sialoglycosphingolipids and a therapeutically active substance, which form micelles with the ability to non-covalently bind antibodies on their surfaces, to form a ternary micellar complex, Sialoglycosphingolipid-Therapeutically active substance-Antibody.

Another object of the present invention is a procedure for obtaining the pharmaceutical composition soluble in aqueous solution, of the ternary micellar complex, Sialoglycosphingolipid-Therapeutically active substance-Antibody of the present invention, comprising the following steps:

(a) solubilize sialoglycosphingolipids, in buffer solution or in a saline solution of pH 4.5, at a concentration above the critical micellar concentration, and heat at a temperature of between 45 and 60 OC, for a time greater than 15 minutes and then allow the solution to stand to form micelles;

(b) add a solvent solution, preferably selected from the set comprising an organic solvent, dimethyl sulfoxide and ethanol, containing a therapeutically active substance;

(c) incubating the mixture of step (b) for incorporation of said therapeutically active substance into the micelles;

(d) dialyzing the micellar solution containing the therapeutically active substance of step (c) against a solution of distilled water or a pharmaceutically acceptable solution having a pH between 4 and 7, so as to remove the solvent;

(e) incubate the micelles resulting from step (d) in the presence of said polypeptide at a pH of between 4 and 7.6, so as to allow the formation of the ternary complex Sialoglycosphingolipids-Therapeutically active substance-Polypeptide, where preferably the mass ratio between sialoglycosphingolipids and polypeptide is between 2:1 and 6:1 (p/p), and said incubation is carried out for a time comprised between 1 and 2 hs at a temperature of at least 45° C.; and preferably the pH of the micelles is in range comprised between pH 4 and 7 wherein said micelles are heated at a temperature comprised between 45 and 65° C. for 1 hour.

Wherein optionally step (g) is performed to lyophilize the sterilized ternary micellar complex obtained in step (f). And further optionally step (h) is performed which consists of resuspending the lyophilizate of step (g) in a pharmaceutically acceptable solution at the time of use.

Furthermore, in the procedure of the invention, in a preferred version, said polypeptides are selected from the set comprised by human polyclonal antibodies, humanized polyclonal antibodies, humanized monoclonal antibodies, polyclonal antibodies of animal origin, monoclonal antibodies of animal origin, chimeric antibodies, fragments thereof and mixtures thereof. As well as said sialoglycosphingolipids are selected from the set comprising monosialogangliosides, disialogangliosides, trisialoganglioside, GM1, LIGA-GM1, GM2, GD1a, GD1b, GT1 and mixtures thereof. And, said therapeutically active substance is selected from the set comprising antitumor drugs, antifungal drugs, hydrophobic drugs, hydrophilic drugs, taxanes, paclitaxel, docetaxel, doxorubicin, amphotericin, prostaglandins, isosorbide dinitrite, prostaglandins, propofol, isosorbide dinitrate, testosterone, nitroglycerin, estradiol, vitamin E, cortisone, dexamethasone and its esters and betamethasone valerate in a molar ratio of therapeutically active substance/sialoglycosphingolipids ranging between 1:2 and 1:100. Wherein, in an alternative embodiment, the present invention comprises as therapeutically active substance docetaxel and as sialoglycosphingolipids: GM1, present in a docetaxel/GM1 molar ratio comprised between 1:10 and 1:100.

And where in another preferred embodiment of realization of the procedure of the invention comprises as therapeutically active substance between 0.1 mg/ml to 6 mg/ml of paclitaxel or docetaxel and between 200 and 300 mg/ml of sialoglycosphingolipids.

And where in another alternative embodiment of the procedure of the present invention, said therapeutically active substance comprises doxorubicin in a doxorubicin/sialoglycosphingolipid molar ratio comprised between 1:2.5 and 1:20.

Another object of the present invention is a pharmaceutical composition soluble in aqueous solution, which is obtainable by means of said procedure of the present invention.

Another object of the present invention is a pharmaceutical composition soluble in aqueous solution, characterized in that it is obtainable in step (d) of said procedure of the present invention.

Another object of the present invention is a procedure for obtaining the pharmaceutical composition soluble in aqueous solution, of the present invention comprising the following steps:

a—solubilize sialoglycosphingolipids: GM1 in acetic-acetate buffer solution or in a saline solution of pH 4.5 always above the critical micellar concentration, and let the solution stand for 24 hs;

b—heat the sialoglycosphingolipid solution at 60° C. for 1 hour, and then allow to cool to room temperature;

c—add an IgG solution at pH 4.5 over the GM1 micelle solution at room temperature and allow to stand for 2 hr;

d—add a DMSO solution containing paclitaxel or docetaxel;

e—dialyze to remove the solvent and unincorporated drug;

f—sterilize this solution by filtration at 0.2 μm;

g—lyophilize.

### DESCRIPTION OF THE FIGURES

FIG. 1.A: Elution profiles obtained by micelle size exclusion chromatography of GM1 and GM1/Ptx.

FIG. 1.B: Elution profile obtained by size exclusion chromatography of IgG.

FIG. 1.C: Elution profiles obtained by size exclusion chromatography of GM1 and GM1/Ptx micelles incubated with IgG.

FIG. 2: Elution profiles obtained by size exclusion chromatography of GM1 micelles incubated with IgG at different pH.

FIG. 3: Elution profiles obtained by size exclusion chromatography of GM1 micelles incubated with IgG at different temperatures.

FIG. 4: Elution profiles obtained by size exclusion chromatography of GM1 micelles incubated with IgG at different times.

FIG. 5: Elution profiles obtained by size exclusion chromatography of GM1 micelles incubated with IgG under different conditions: (-) IgG alone, ( [symbol] ) Micelles of GM1 (20 mg/ml) in buffer pH 4.5 were heated to 45° C. and then IgG is incorporated, ( [symbol] ) Micelles of GM1 (20 mg/ml) in buffer pH 4.5 were heated to 60° C., then allowed to cool and IgG is incorporated.

FIG. 6: GM1 micelles incubated with increasing amounts of IgG.

FIG. 7. A: Elution profiles obtained by size exclusion chromatography of GM1 micelles incubated with anti-Rub IgG antibody.

FIG. 7.B: Native SDS-PAGE electrophoresis of elution aliquots 1 to 7 of the GM1-IgG anti-Rub sample (lane 2-8). Lane 1 corresponds to the anti-Rub IgG standard.

FIG. 7.C: Elution profiles obtained by size exclusion chromatography of GM1 micelles incubated with anti-HBS IgG antibody.

FIG. 7.D: Native SDS-PAGE electrophoresis of elution aliquots 1 to 7 of the GM1-IgG anti-HBS sample (lane 2-8). Lane 1 corresponds to the anti-HBS IgG standard.

FIG. 8.A: Elution profiles obtained by size exclusion chromatography of GM1-IgG micelles incubated with different amounts of Albumin.

FIG. 8.B: SDS-PAGE electrophoresis (without 2-mercaptoethanol) of GM1/IgG micelles incubated with different amounts of albumin (Alb.) at pH 4.5. Lanes 1-2—for IgG and Alb. standards; Lanes 3-10 for fractions eluted from the size exclusion column (a): GM1—IgG micelles with 2.2 mg·ml-1 of Alb. (b): GM1—IgG micelles with 4.4 mg·ml-1 of Alb. (c): GM1—IgG micelles with 8.8 mg·ml-1 of Alb.

FIG. 9.A: Elution profiles obtained by size exclusion chromatography of GM1 and GM1/Doxo micelles.

FIG. 9.B: Elution profile obtained by size exclusion chromatography of IgG.

FIG. 9.C: Elution profiles obtained by micelle size exclusion chromatography of GM/IgG and GM1/IgG/Doxo.

FIG. 10.A: Elution profiles obtained by micelle size exclusion chromatography of GM1/IgG/Doxo (A) and GM1/Doxo/IgG (B). Absorbance 280 nm.

FIG. 10.B: Elution profiles obtained by micelle size exclusion chromatography of GM1/IgG/Doxo (A) and GM1/Doxo/IgG (B). Absorbance 490 nm.

FIG. 11: Incorporation of IgG into GM1 micelles.

### DETAILED DESCRIPTION OF THE PRESENT INVENTION

### Definitions

For the purposes of the present invention the following terms or expressions are defined:

The terms “therapeutically active substance”, “drug”, “active principle”, “active agent” and “bioactive substance”, have the same meaning and are used interchangeably.

In the present invention, the expression: polypeptide refers to polypeptides or proteins capable of directing micelles specifically to a target tissue, organ or cell, for example, by recognizing a surface molecule present on said target tissue, organ or cell. Particularly, said polypeptide may be an immunoglobulin, an antibody or fragments thereof.

The terms immunoglobulin, antibody, Ac. and Ig are used interchangeably and have the same meaning as they normally have in the prior art. In the present invention the “polypeptide” of the present invention may be an immunoglobulin or fragments or variants thereof.

The terms micelles or nanomicelles, are used interchangeably and refer to micellar structures consisting of sialoglycosphingolipids and wherein said sialoglycosphingolipids may be selected from the set comprising monosialogangliosides, disialogangliosides, trisiaiogangliosides and mixtures thereof; wherein said micellar structure may contain a therapeutically active substance.

The present invention highlights four differences between the ternary micellar complex, the main object of the present invention, from other attempts taught in the present state of the art, related to the incorporation of mono or polyclonal antibodies on ganglioside micelles and other structures (liposomes or nanoparticles) that may contain drugs inside. The first difference to be highlighted is compared to those ganglioside micelles that are coated with serum proteins, for example, albumin, since the latter do not have the capacity of specific targeting to the different types of tissues as can be obtained by the application of specific mono or polyclonal antibodies on these ganglioside micelles as mentioned in the present invention. Particularly in this application a series of procedures are described which are applied on the ganglioside micelle, or antibodies, which allow the incorporation of the same in a stable manner on the ganglioside micelles.

A second difference is that the antibodies incorporated in the micelle are not covalently bound as occurs in the other procedures where antibodies are incorporated on different nanoparticles using crosslinking agents that can affect both the antibody used and the nanoparticle. As mentioned above, the use of these crosslinking agents is an undesirable condition in this type of coupling, since they can produce changes in the biological activity of antibodies as well as in other proteins with biological activity.

Another difference is the low complexity of the procedures involved in obtaining the complex. As can be seen in the background of the state of the art mentioned on the procedures used to incorporate the antibodies, all of them present several different steps to reach the Ac-nanoparticle complex, but the most important thing about these procedures is that, in all cases, the use of different chemical crosslinking agents is mentioned to covalently bind the antibody to different parts of the nanoparticles used.

Finally, a fourth difference lies in the size of the nanoparticles on which the antibodies are incorporated, generating complexes of a size between 10 and 60 nm.

The inventors of the present invention have developed a new formulation based on stable micellar structures of sialoglycosphospholipids that overcomes the problems mentioned above, since they allow incorporation in a non-covalent way on their surface antibodies with the capacity to retain in a specific way these micelles in specific sites or tissues where they can release those drugs that can be incorporated in these micelles.

The present invention is relative to a water-soluble pharmaceutical composition based on sialoglycosphingolipids, and a therapeutically active substance, which can incorporate non-covalently mono- or polyclonal antibodies to perform site-specific targeting of the micelle. This combination of sialoglycosphingolipids, therapeutically active substance and antibody is called a ternary micellar complex.

In one manifestation of the present invention, the sialoglycosphingolipids comprised in the ternary micellar complex are selected from the set comprising monosialogangliosides, disialogangliosides, trisialogangliosides and mixture thereof. In a more preferred embodiment, the monosialoganglioside is selected from the group consisting of GM1, GM1 referred to as LIGA (GM1 in which a classical fatty acid of GM1 has been replaced by an acetyl group) and GM2 or a mixture thereof, the disialoganglioside is selected from the group consisting of between GD1a and GD1b or a mixture thereof; and the trisialoganglioside is selected from the set comprised by GT1 or a mixture thereof.

In another embodiment of the present invention, the therapeutically active substance comprised in the ternary micellar complex, sialoglycosphingolipid-therapeutically active substance-polypeptide, is selected from the set comprised by antitumor drugs, antifungal drugs, hydrophobic drugs, hydrophilic drugs, taxanes, paclitaxel, docetaxel, doxorubicin, amphotericin, prostaglandins, isosorbide dinitrite, prostaglandins, propofol, isosorbide dinitrate, testosterone, nitroglycerin, estradiol, vitamin E, cortisone, dexamethasone and its esters, and betamethasone valerate.

In another embodiment of the present invention, the antibodies comprised in the ternary micellar complex, which coat non-covalently associates, the micellar structures are selected from the complex comprising polyclonal human antibodies, polyclonal humanized antibodies, monoclonal humanized antibodies, polyclonal antibodies of animal origin, monoclonal antibodies of animal origin, chimeric antibodies, fragments thereof and mixtures thereof.

In another preferred embodiment, the pharmaceutical compound is soluble in aqueous solution, composed of water-soluble gangliosides, comprising at least one compound selected from among the sialoglycosphingolipids (mono-, di- and tri-sialo-ganliosides, GM, GD or GT respectively), which form a micellar structure that has the ability to be coated with at least one biologically active mono- or polyclonal antibody, or fragments or variants thereof, non-covalently bound to said micellar structure, and which may furthermore contain one or more therapeutically active substances. In a preferable embodiment, the monosialogangliosides are selected from the group consisting of GM1, GM1 referred to as LIGA (GM1 in which a classical fatty acid of GM1 has been replaced by an acetyl group) and GM2 or a mixture thereof, the disialogangliosides are selected from the group between GD1a and GD1b or a mixture thereof; and the trisialoganglioside is selected from the set comprised by GT1 or a mixture thereof. In another preferred embodiment, said active substance is selected from the set composed of antitumor drugs, antifungal drugs, hydrophobic drugs, hydrophilic drugs, taxanes, paclitaxel, docetaxel, doxorubicin, amphotericin, prostaglandins, isosorbide dinitrite, prostaglandins, propofol, isosorbide dinitrate, testosterone, nitroglycerin, estradiol, vitamin E, cortisone, dexamethasone and its esters and betamethasone valerate. In another preferable embodiment, the antibodies coating the surface of the micellar structure are selected from the complex composed of polyclonal human antibodies, polyclonal humanized antibodies, monoclonal humanized antibodies, polyclonal antibodies of animal origin, monoclonal antibodies of animal origin, chimeric antibodies, fragments thereof and mixtures thereof.

In another preferred embodiment, the pharmaceutical compound of the present invention is a sterile and translucent injectable composition.

The sialoglycosphingolipid micelles of the present invention, when heated to temperatures of 55-60° C., undergo a change at the level of their polar head consisting in the extrusion of water molecules between the oligosaccharide chains, which leads to an increase in the hydrophobicity of the medium. This increase in hydrophobicity is what allows the antibodies to intercalate in the ganglioside micelle, doing so by an interaction through their Fc fraction, that fraction of the immunoglobulin that does not have specific recognition, leaving on the surface the segment known as Fab, which presents the biological activity of specific antigen recognition.

A relevant factor of this invention is that the micelles of monosialo and disialogangliosides or their mixtures with anticancer drugs such as Paclitaxel (Ptx) or Doxorubicin (Doxo), retain their capacity to interact and incorporate on their surface the immunoglobulins or mono or polyclonal antibodies by means of the procedure of the present invention mentioned previously. In this procedure, the hydrophobic interaction between the GM1 micelles and the immunoglobulins occurs spontaneously and non-covalently, forming a new stable micelle-antibody or micelle-drug-antibody complex. Said ternary complex sialoglycosphingolipids-therapeutically active substance-polypeptide has special properties due to the presence of said polypeptides (e.g. antibodies) on the surface, non-covalently associated, which allow to concentrate said micelles loaded with the therapeutically active substance (e.g. oncological), specifically in the target tissue recognized by the selected antibody.

In one embodiment, monosialoganglioside, disialoganglioside and trisialoganglioside complexes or mixtures thereof, containing Ptx or Doxo, can be coated with polyclonal or monoclonal antibodies or fragments thereof, which can be delivered to humans directly into the bloodstream via injection, or by prior insertion into transfusion bags containing human albumin, whole human serum or whole human plasma.

The extremely low CMC of the gangliosides (10-8′10-9M), added to the already mentioned stabilization given by the loading of the active principles in the structure, would allow a longer half-life in circulation.

It is therefore, one of the objects of the present invention, a formulation presenting a carrier, or support, composed of sialoglycosphingolipid micelles, more specifically by monosialoyl, disialoyl or trisialoglycosphingolipids, or a mixture of them over their CMC, which allow incorporating ligand-binding polypeptides in a non-covalent way, such as antibodies, forming highly soluble complexes that can also contain hydrophobic or highly cytotoxic drugs.

In another preferred embodiment, the pharmaceutical compound of the invention is a composition adapted to be administered to the patient in injectable, sterile and translucent form. In a particular manner, the water-soluble pharmaceutical composition of the invention is lyophilized. In that case, the composition is reconstituted with a solvent selected from the group consisting of distilled water, saline solution (NaCl 0.9%), phosphate buffered saline solution (PBS), distilled water containing 5% dextrose and saline solution with 5% dextrose. Thus, there are also particular objects of the present invention, a lyophilized, water-soluble and sterile pharmaceutical composition, which can be resuspended so that antitumor drugs such as Docetaxel (Dtx) or Ptx have a final concentration comprised between 0.1 and 10 mg/ml, more preferably between 1 and 6 mg/ml; and still more preferably between 3 and 6 mg/ml.

It is also an object of the present invention that the micelle formulations contain, on their surface, therapeutically active proteins such as non-covalently bound mono- or polyclonal antibodies, in a final concentration comprised between 0.1 and 2 mg of antibodies per 5 mg of sialoglycosphingolipid micelle. In view of the above, it should be noted that the ligand-binding polypeptide can have the function of directing and concentrating the sialoglycosphingolipid micelle, but it can also have therapeutic action, as is the case of Trastuzumab®.

More preferably still, the pH of the micelles is comprised in the range of between 4 and 7. In a still more preferred embodiment, the pH of the nanomicelles is comprised in the range of between 4 and 6.

Particularly, in the soluble pharmaceutical compositions of the invention, the nanomicelles possess an average size smaller than 100 nm, more particularly still, smaller than 50 nm, preferably, between about 5 nm and about 40 nm and, still more preferably, between about 10 nm and about 30 nm.

In a particularly preferred embodiment of the invention, the water-soluble pharmaceutical composition comprises ganglioside nanomicelles coated non-covalently with human serum immunoglobulins, polyclonal human serum immunoglobulins G (IgG), monoclonal humanized serum immunoglobulins G (IgG), more preferably with immunoglobulins type G (IgG) directed against specific antigens present on the surface of tumor cells, or endothelial cells. More particularly still, they are coated with polyclonal or monoclonal immunoglobulins type G (IgG), in a GM1:IgG mass ratio between 2:1 and 10:1.

In a particularly preferred embodiment of the invention, the water-soluble pharmaceutical composition comprises coated ganglioside nanomicelles non-covalently associated with polyclonal human IgG-type immunoglobulins or humanized monoclonal IgG-type immunoglobulins directed against tumor or endothelial cell surface antigens.

In preferred embodiments of the invention, the water-soluble pharmaceutical composition of antibody-coated sialoglycosphingolipid micelles contains drugs such as Ptx or Dtx. Preferably, it comprises Ptx or Dtx in a drug: gangliosides molar ratio of between 1:10 and 1:100. More preferably still, it comprises between 0.1 mg/ml and 6 mg/ml of Ptx or Dtx and between 4 mg/ml and between 200 and 300 mg/ml of glycosphingolipids.

In another particularly preferred embodiment, the water-soluble pharmaceutical composition of ganglioside micelles coated with antibodies of the present invention containing Doxo in a Doxo: ganglioside molar ratio comprised between 1:1 and 1:50.

In another particularly preferred embodiment, the water-soluble pharmaceutical composition of ganglioside micelles coated, in a non-covalent manner, with antibodies of the present invention containing at least two therapeutically active substances, wherein one of them is Doxo, wherein the second is selected between Ptx and Dtx in a molar ratio for each of drug:drug:ganglioside comprised between 1:10 and from 1:100.

Another object of the present invention comprises a procedure for obtaining the pharmaceutical composition soluble in aqueous solution of the present invention. In regard to the loading of the therapeutically active substance into the micelles, for obtaining the pharmaceutical compound soluble in aqueous solution object of the present invention, it can be carried out as described below: The micelles obtained are incubated in the presence of one tenth of their volume (1/10 vol/vol) with organic solvents capable of dissolving the hydrophobic therapeutically active substance. The samples are incubated at a temperature between 4 and 8° C. for at least 4 hs. The organic solvent is then removed from the micelle preparation obtained by a dialysis process and finally centrifuged between 15,000 and 30,000×g for 15 minutes in order to remove the therapeutically active substance that would not have been incorporated in the micelles. The transparent aqueous formulation of micelles obtained previously is incubated under two different experimental conditions.

In the specific case of immunoglobulin loading, it is necessary to introduce a series of modifications in the incubation medium of the micelles with the IgG, so that their association to the micellar structure can occur, since, under classical pH and temperature conditions, the immunoglobulins are not incorporated into the ganglioside micelles. In this sense, to obtain the maximum incorporation of the IgG into the micelles, the system must be incubated at acid pH, preferably at a pH of 4.5, where the IgG increase the exposure of their hydrophobic domains, and incubation at a temperature between 40 and 65° C. is also necessary. In the case of temperature effect, it can be done in two ways: (1) incubating the antibody and micelles, for example, of GM1, at 45° C., which favors the incorporation of IgG into the micelles, without essentially affecting the activity of the antibodies and (2) only heating the sialoglycosphingolipid micelles to 65° C., then allowing the solution to cool and finally the antibodies are incorporated at room temperature, maintaining pH 4.5.

Thus, the present invention also refers to a procedure for obtaining micelles superficially coated, in a non-covalently manner, by at least one polypeptide, specifically antibodies directed against specific antigens, which may contain in their interior at least one therapeutically active substance.

In a preferred embodiment, the procedure can be summarized as follows: (a) solubilizing sialoglycosphingolipids, in a buffer solution or in a saline solution of a pH 4.5, at a concentration above the critical micellar concentration, and heating at a temperature between 45 and 60 OC, for more than 15 minutes and then allowing the solution to stand to form micelles; (b) adding a solvent solution containing a therapeutically active substance; (c) incubating the mixture of step (b) for the incorporation of said therapeutically active substance into the micelles; (d) dialyze the micellar solution containing the therapeutically active substance of step (c) against a solution of distilled water or a pharmaceutically acceptable solution having a pH between 4 and 7, so as to remove the solvent; (e) incubating the micelles resulting from step (d) in the presence of said polypeptide at a pH between 4 and 7.6, so as to allow the formation of the ternary complex Sialoglycosphingolipid-therapeutically active substance-polypeptide; (f) sterilizing the aqueous and clear solution obtained from the previous step. In a more preferred embodiment, the procedure for obtaining the aqueous solution-soluble pharmaceutical composition of the present invention further comprises a freeze-drying step of the sterilized ternary micellar complex obtained in step (f).

Alternative ways of achieving non-covalent association between the micelle and the polypeptide are described below: the monosialoganglioside GM1 or GM2, or the disialogangliosides GD1a and GD1b or their mixtures are dissolved in distilled water by gentle agitation and then allowed to stand at a temperature between 4 and 8° C. for at least 24 hs, after which the micelles and antibodies are incubated at pH 4.5 in a micelle/antibody ratio by weight between 1/0 25 and 1/0 5:

1—Micelles and antibodies are incubated at pH 4.5 at a micelle/antibody ratio by weight between 1/0.25 and 1/0.5, and allowed to stand for 30 min, then heated at 45° C. for a time interval between 30 and 60 min, in order to ensure interaction and incorporation of the antibody into the micelle.

Finally, it is passed through a molecular filtration column (Sephadex G200) to remove the antibody that has not been incorporated into the micelle, or

2—The GM1 micelle at pH 4.5 is heated at 60° C. for 30 to 60 minutes and then the solution is allowed to cool. The antibodies are then added in a GM1-IgG weight ratio of I/0.25 or I/0.5 and allowed to stand for 1 hour. Finally, the material is passed through a molecular filtration column (Sephadex G200) to remove immunoglobulin that has not been incorporated into the micelle.

Hereunder, we describe in detail the procedures for the incorporation of antibodies on the surface of ganglioside micelles, as well as the incorporation of different drugs inside the micelle-antibody complexes:

PROCEDURE 1—It comprises the following steps:

(a) Solubilize the gangliosides in distilled water or in a saline solution with a pH between 3 and 7, always above the critical micellar concentration, leaving the solution to stand at 4° C. for at least 24 hs.

(b) Incubate the product obtained in step (a) with the bioactive agent, in this case the mono or polyclonal antibodies as previously defined.

(c) Filtration of the material obtained in step (b) through a molecular filtration column (Sephadex G200) in order to remove the IgG that has not been incorporated in the micelle.

(d) In the case of drug incorporation into the micelles with antibodies, it can be carried out as follows. Initially, the GM1 micelles are heated to 65° C. and then cooled, the drugs are incorporated into the micelle as previously mentioned, and finally the antibodies at pH 4.5, thus forming the ternary complex micelle-drug-IgG.

(e) Dialyze the micellar solution obtained in step (d) against a solution of distilled water or a pharmaceutically acceptable solution having a pH between 3 and 7, for 24 h at a temperature between 4 and 8° C., to completely remove the organic solvent.

(f) Finally the solutions obtained in both procedures 1 and 2, can be sterilized in liquid form, by filtration through 0.1 or 0.2 μm and packaged directly for its use, or proceed to lyophilize them in a new presentation.

(i) Resuspend the lyophilized micelles in a pharmaceutically acceptable solution so that they can be administered in intravenous injectable form for the treatment of the pathology in question.

The amount of the various therapeutically active substances incorporated (Ptx, Dtx, Doxo) and the concentration of the polypeptides in the ganglioside nanomicelles can be determined using either spectroscopic techniques or suitable chromatographic techniques, such as high-pressure liquid chromatography (HPLC) or electrophoresis on polyacrylamide gels.

### EXAMPLES

### Example 1: Elution Profile of IgG Incorporated in GM1 Micelles Alone or Previously Loaded with Ptx by Sephadex G-200 Column

Micelles of GM1 and GM1-Ptx were prepared in acetic/acetate buffer pH=4.5, containing 20 mg/ml GM1, and a Ptx-GM1 molar ratio of 1/20. These micelles were incubated in the presence of a fixed amount of human IgG (5 mg/ml) for 1 hour at a temperature of 37° C.

After incubation, the samples were centrifuged at 15,000×g for 15 minutes and subjected to molecular filtration using a Sephadex G200 column to separate the GM1-Ptx-IgG complexes from the free IgG. The volume eluted through the column was collected in fractions of 1 ml each. Finally, the elution profile of IgG incorporated in soluble form in the GM1 or GM1/Ptx micelle was determined by absorbancla at 280 nm against an IgG standard.

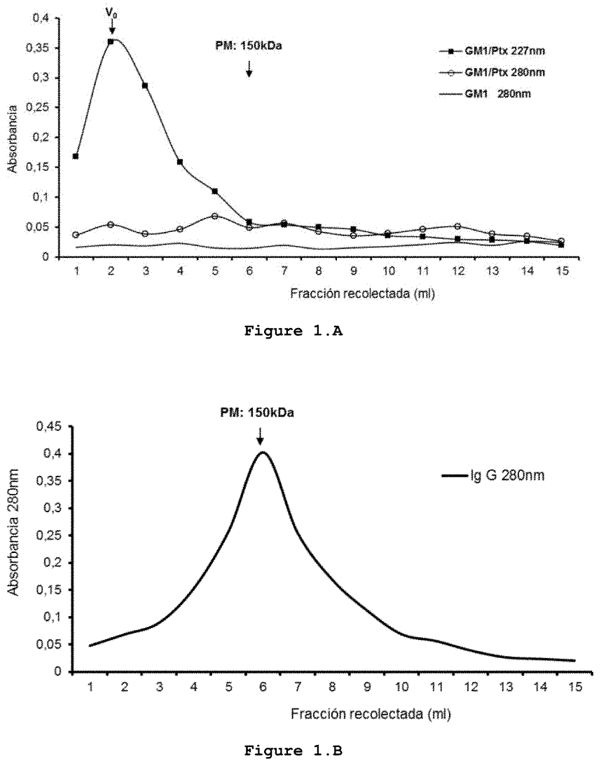

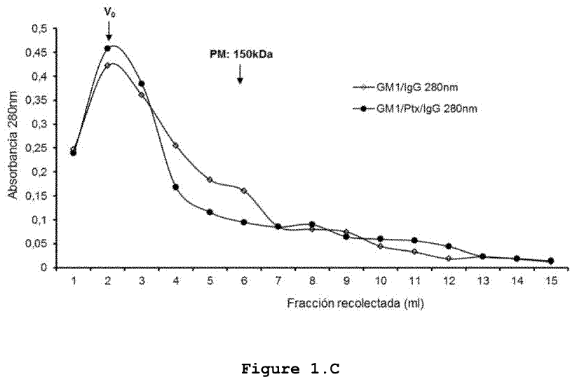

The GM1 micelle as well as GM1/Ptx micelles have no absorbance at 280 nm. The increase in absorbance at 227 nm in the collected fractions 1-4 of GM1 or GM1/Ptx micelles coincides with the column elution volume (Vo) determined with Dextran Blue. This profile shows that Ptx elutes together with the micelle and that the complex corresponds to a PM greater than 250 kDa (see FIG. 1.A). As shown in FIG. 1.B the increase in absorbance from fraction 5 to 9 corresponds to the PM of 150 kDa corresponding to an immunoglobulin. In FIG. 1.C a shift to the left can be seen in the elution profile at 280 nm of IgG incubated with GM1 or GM1/Ptx micelles with respect to standard IgG, which is evidence that there is an interaction between IgG incubated with GM1 or GM1/Ptx micelles, as well as that the presence of Ptx loaded in the micelle does not modify the interaction of IgG with the micelle.

### Example 2: Effect of pH on IgG Incorporation into GM1 Micelles Loaded with Ptx

Ptx is loaded onto GM1 micelles at ratios as previously described in Example 1. Incorporation of a fixed amount of IgG (10 mg/ml) onto Ptx-loaded GM1 micelles is performed at pHs 3.6-4.5 and 7, for 1 hour at 37° C. After incubation, samples were centrifuged at 15,000×g for 15 minutes and subjected to molecular filtration on Sephadex G 200 separating GM1-Ptx-IgG complexes from free IgG. Finally, the elution profile of IgG incorporated in the GM1 micelle, at different pHs, was determined by absorbance at 280 nm against an IgG standard (10 mg/ml).

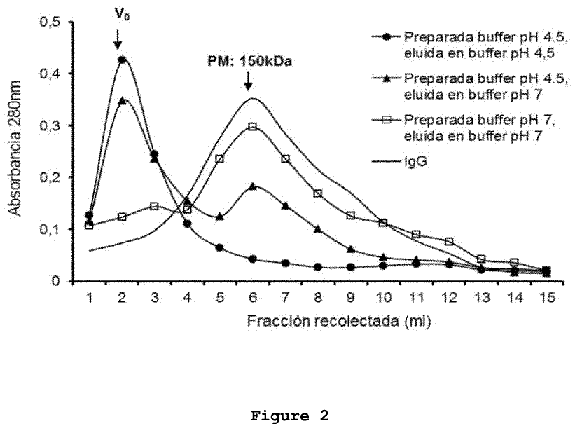

The pH of the medium influences the interaction between GM1 micelles and IgG. The amount of IgG that appears forming the GM1-Ptx-IgG complex (fractions 1-4) increases at acidic pH (4.5) with respect to neutral (7). The formation of insoluble aggregates at pH<4 does not allow elution through the column demonstrating instability in the complexes formed or denaturation of the IgG. Fractions 1-4 correspond to column elution volume (Vo). Fractions 5-9 correspond to the 150 kDa PM of immunoglobulin G (see FIG. 2).

### Example 3: Effect of Temperature on IgG Incorporation into Ptx-Loaded Monosialoganglioside Micelles

Incorporation of IgG into the micelles of Example 1 was carried out at pH 4.5 for 1 hour at temperatures of 4, 25, 37 and 45° C. After incubation, samples were centrifuged at 15,000×g for 15 min and subjected to molecular filtration on Sephadex G200 to separate GM1-Ptx-IgG complexes from free IgG. Finally, the elution profile of IgG incorporated into the GM1 micelle was determined by absorbance at 280 nm against an IgG standard (5 mg/ml).

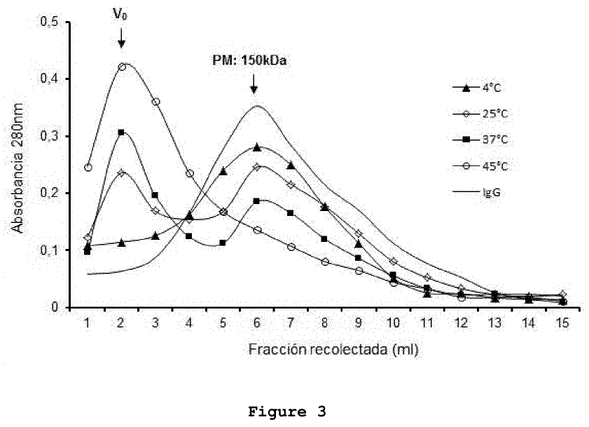

Temperature regulates the interaction of the GM1/Ptx complex with the IgG added to the medium (see FIG. 3). As temperature increases, the amount of IgG that appears forming the GM1-Ptx-IgG complex increases. At higher temperature, higher absorbance is observed in fractions 1-4 corresponding to the elution volume of the column (Vo). Fractions 5-9 correspond to the PM of IgG which is 150 kDa.

### Example 4: Effect of Incubation Time on the Incorporation of IgG into GM1/Ptx Micelles

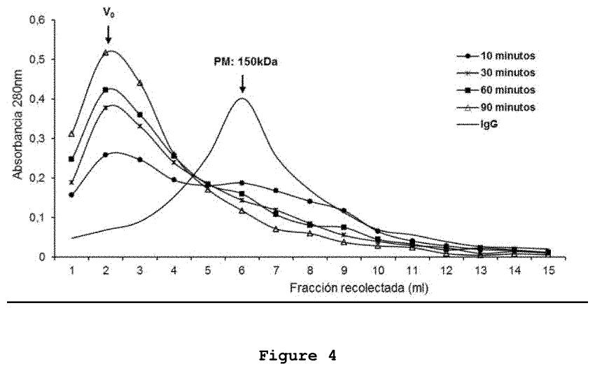

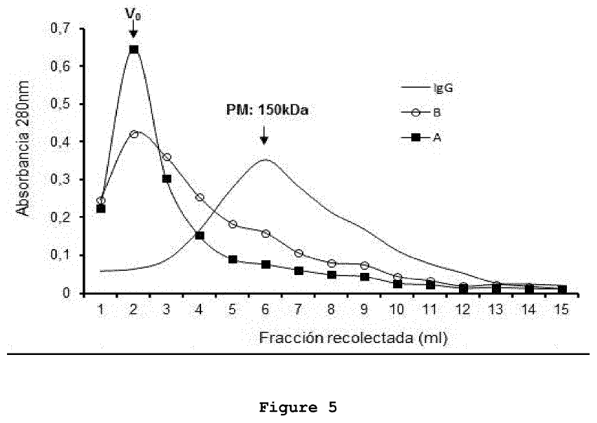

The incorporation of IgG (5 mg/ml) on the micelles obtained in Example 1 (20 mg/ml) was performed by incubating the mixture at pH 4.5 and at a temperature of 45° C., at different times (see FIG. 4). After incubation, the samples were centrifuged at 15,000×g for 15 minutes and subjected to molecular filtration on Sephadex G 200 in order to separate the GM1-Ptx-IgG complexes from the free IgG. The volume eluted through the column was collected in fractions of 1 ml each. Finally, the elution profile of IgG incorporated in the GM1 micelle at different incubation times was determined by absorbance at 280 nm against an IgG standard. It was observed that, as the incubation time increases, the amount of IgG forming the GM1—Ptx-IgG complex increases (see FIG. 4). The longer the incubation time, the higher absorbance appears in fractions 1-4 (column elution volume (Vo)). Fractions 5 to 9 correspond to the PM of an IgG (150 kDa).

### Example 5: Effect of Temperature on the GM1 Micelle on its Capacity for IgG Incorporation

Micelles of GM1 (20 mg/ml) in buffer pH 4.5 were heated at 45° C. or 60° C. for the time of 1 hour. They were allowed to stand until room temperature (20° C.) was reached. A fixed amount of IgG (5 mg/ml) was incorporated at 20° C. for 1 hour. Samples were centrifuged at 15,000×g for 15 minutes and molecular filtration was performed on Sephadex G 200 to separate GM1-IgG complexes from free IgG. Finally, the elution profile of IgG incorporated in preheated GM1 micelles at the different temperatures was determined by absorbance at 280 nm against an IgG standard.

### Example 6: Immunoglobulin Loading on GM1 Ganglioside Micelles Loaded with PacSitaxeS

The micelles obtained in Example 1, loaded with Ptx at a concentration of 5 mg/ml, were then incubated in the presence of increasing amounts of IgG to reach a final concentration of 1, 25; 2.5; 5; 10 and 15 mg/ml. Incubations were performed at a temperature of 20° C. for 1 hour. The samples were then centrifuged at 15,000×g for 15 minutes and filtered over Sephadex G 200 to separate the GMi-Ptx-IgG complexes from the free IgG. Finally, amount of the IgG incorporated in the GMi-Ptx micelle was determined by absorbance at 280 nm against an IgG standard.

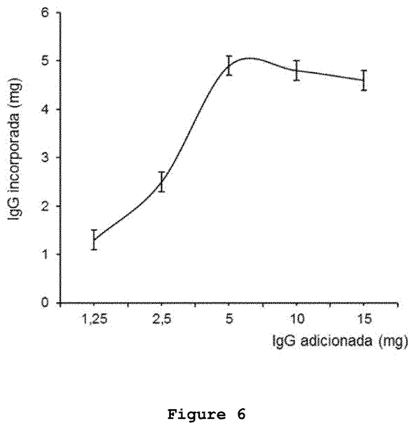

It was observed that in the presence of a fixed amount of GMi (20 mg/ml), the increase in the amount of IgG produced an increase in the amount of the GM1-Ptx-IgG complex until saturation was reached at 5 mg/ml (see FIG. 6).

### Example 7: Elution Profile of Anti-Rubella and Anti-Hepatitis B Monoclonal Antibodies Incorporated in GM1 Micelles

Micelles of GM1 (40 mg/ml) in buffer pH 4.5 were heated at 55° C. for 1 hour. The resulting GM1-Ptx micelles were incubated in the presence of a fixed amount of anti-rubella (Anti-Rub 7.5 mg/ml) or anti-hepatitis b (Anti-HBS 1 10 mg/ml) monoclonal antibody. Antibody alone controls were also incubated at pH 4.5 for 1 hour at 20° C. The pH was then adjusted again to 7 with NaOH. After incubation, samples were centrifuged at 15,000×g for 15 minutes and subjected to molecular filtration on Sephadex G 200 to separate monoclonal GM1/Acs complexes from free Acs. The volume eluted through the column was collected in 1 ml fractions. The elution profile of the monoclonal antibody incorporated into GM1 micelles was determined by absorbance at 280 nm against the antibody used as a control. To confirm the correlation between the elution peaks at 280 nm with the presence of the proteins (IgG), electrophoresis was performed on 7.5% polyacrylamide gels with SDS.

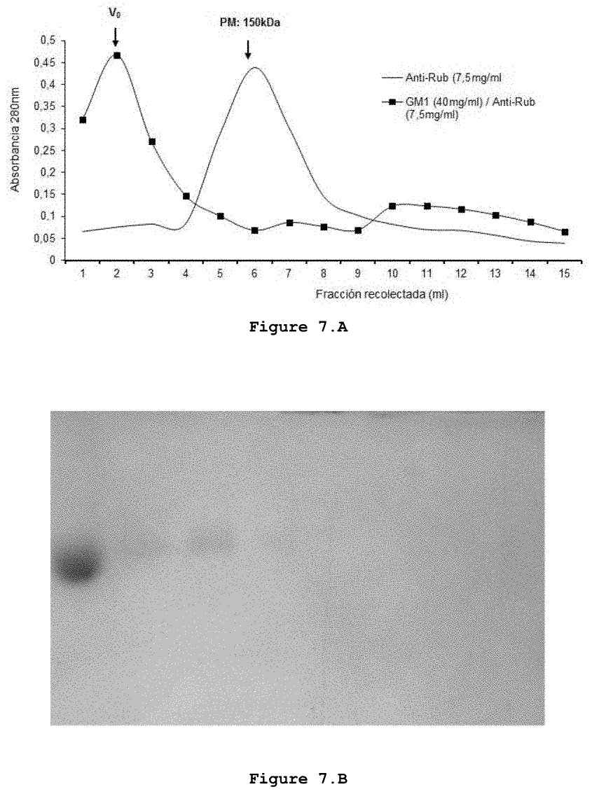

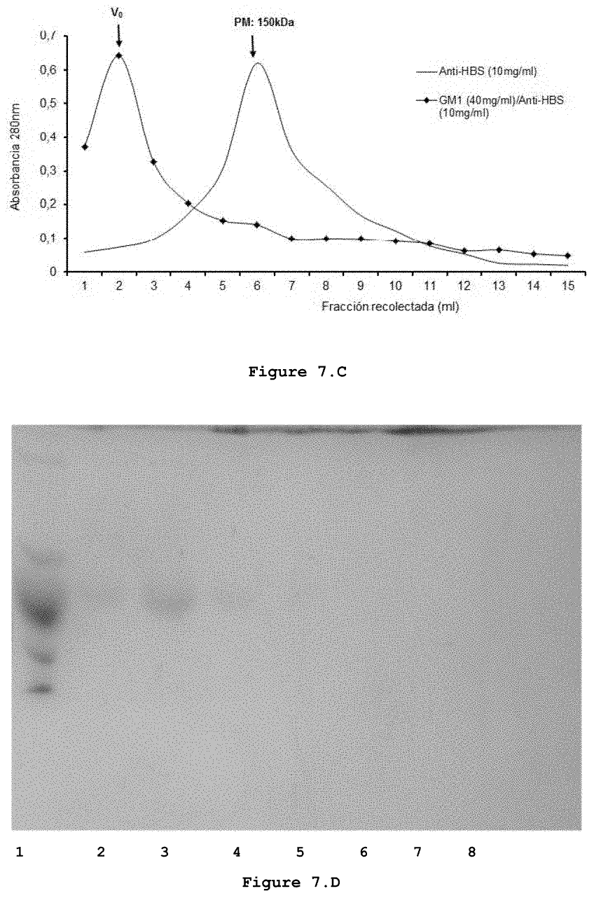

In the presence of GM1 micelles, there is a shift in the elution profile of the Anti-Rub or Anti-HBS monoclonal antibody relative to the standard antibody (see FIGS. 7.A and 7.C). The mixtures elute in the first fractions (1-4) which corresponds to the elution volume (VO) of the column (PM greater than 250 kDa.).

### Example 8: Determination of the Biological Activity of Anti-Rub and Anti-HBS Monoclonal Antibodies Incorporated into Ptx-Loaded GM1 Micelles

The incorporation of the anti-rubella (Anti-Rub) and anti-hepatitis b (Anti-HBS) monoclonal antibodies into GM1 micelles was performed following the procedure described in Example 7. The determination of the immunogenic activity was carried out by the electrochemiluminescence immunoassay technique “ECLIA”. For anti-Rubella (Anti-Rub) antibodies, the SIEMENS IMMULITE 2000 test was used, based on a commercially available two-step solid-phase quantitative chemiluminescent chemiluminescent immunoassay for in vitro diagnostic use.

Antibodies (Anti-HBS) against hepatitis B surface antigen (HBsAg) were quantitatively determined by sandwich chemiluminescent immunoassay (ELECSYS anti-HBs kit from ROCHE). Tables 1 and 2, show the results of the electrochemiluminescence assays (ECLIA) obtained for Anti-Rub or Anti-HBs antibodies respectively, as well as the amounts (mg) of GM1 and antibodies used as the dilutions performed for each case. The assays were performed in duplicate.

TABLE 1
A: IMMULITE 2000 test for the quantitative determination of IgG antibodies against
rubella virus. B: ELECSYS anti-HBs test for the quantitative determination of IgG antibodies
against hepatitis B surface antigen.

| A | | B | |
|---|---|---|---|
| Anti-rub immulite | | A-HBS Elecsys | |
| sample | Unit mUl/ml | sample | Unit mUl/ml |
| Control (+) | 11.6 ± 2.3 | Control (+) | 31.2 ± 1.7 |
| equipment | | equipment | |
| 10 ul Anti-Rub in | 11.3 ± 4.0 | 10 ul Anti-HBS | 31.8 ± 3.4 |
| buffer | | in buffer | |
| 4 mg GM1 + | 11.1 ± 4.2 | 4 mg GM1 + | 19.6 ± 4.1 |
| 10 ul Anti-Rub | | 10 ul Anti-HBS | |
| 8 mg GM1 + | 13.7 ± 3.0 | 8 mg GM1 + | 17.7 ± 1.6 |
| 10 ul Anti-Rub | | 10 ul Anti-HBS | |
| 4 mg GM1 + | 33.3 ± 6.1 | 4 mg GM1 + | 59.3 ± 8.2 |
| 30 ul Anti-Rub | | 30 ul Anti-HBS | |

### Example 9: Regulation of Albumin Presence on IgG Incorporation into GM1-Ptx Micelle

Micelles of GM1 (20 mg/ml) in buffer pH 4.5 were heated at 55° C. for 1 hour. They were allowed to stand until room temperature (20° C.) was reached. Incorporation of a fixed amount of IgG (5 mg/ml) in the presence of increasing amounts of purified human serum albumin (2.2, 4.4 and 8.8 mg/ml) onto the GM1 micelles (20 mg/ml), was carried out by incubating the mixture at pH 4.5 and at a temperature of 20° C. for 1 hour.

After incubation, the samples were centrifuged at 15,000×g for 15 minutes and subjected to molecular filtration with Sephadex G 200 to separate the various complexes formed from the free proteins. Finally, the elution profile of IgG incorporated in GM1/Ptx micelles in the presence of Albumin was determined by absorbance at 280 nm against standard. As a complement, 7.5% polyacrylamide gels were run by SDS-PAGE technique without 2-mercaptoethanol to confirm the presence of Albumin and native IgG.

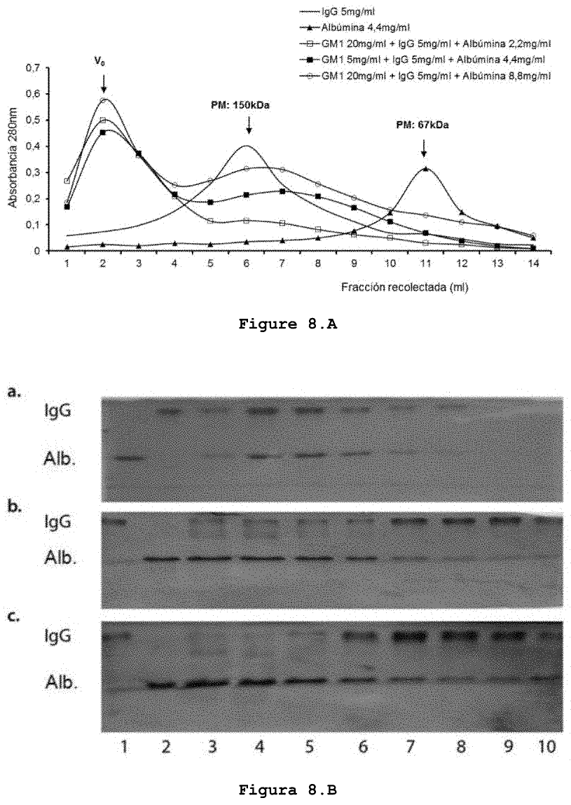

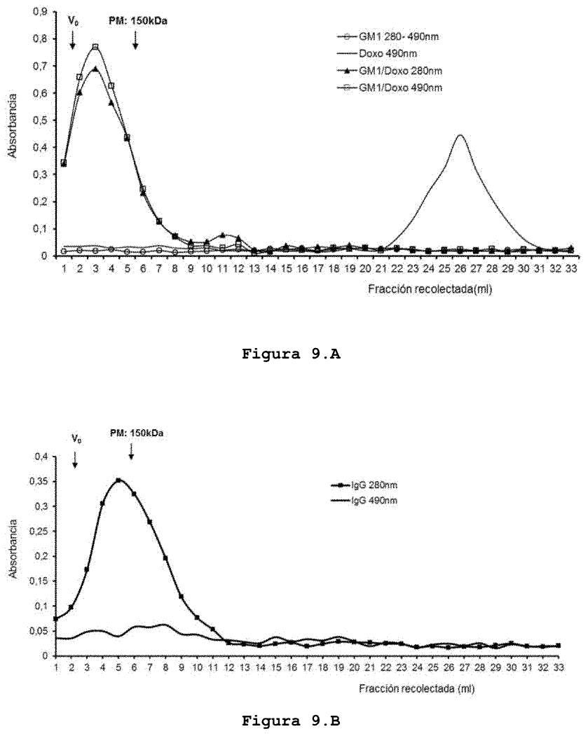

As the amount of Albumin added increases, there is an increase in the population of PM: 150 kDa (fraction 5-9) corresponding to free IgG (see FIG. 8.A). B show the changes in the elution profile of a fixed amount of IgG (20 mg) as a function of increasing amounts of Albumin from 2.2 to 4.4 and 8.8 mg, respectively (see FIG. 8.B: a, b, c). A decrease in the amount of IgG eluting in the first fractions (1-4), corresponding to the exclusion volume of the column (VO), was observed. In parallel, an increase in the amount of IgG was observed in the fractions (5-8) representing the fractions where free IgG elutes (PM: 150 kDa).

### Example 10: Sephadex G-200 Elution Profile of Sas IgG Incorporated in GM1 or GM1-Doxorubicin Micelles

GM1 and GM1-Doxo micelles were prepared in acetic/acetate buffer pH 4.5, containing 20 mg/ml GM1, and a Doxo-GM1 molar ratio of 1/10. These micelles were incubated in the presence of a fixed amount of human IgG (5 mg/ml) for 1 hour at a temperature of 45° C. After incubation, samples were centrifuged at 15,000×g for 15 minutes and subjected to filtration on Sephadex G 200 to separate GM1-Doxo-IgG complexes from free IgG. The volume eluted from the column was collected in 1 ml fractions.

The elution profile of IgG incorporated in soluble form in the GM1 or GMI/Doxo micelle was determined by measurement of absorbance at 280 nm against an IgG standard. Also, the elution profile at 490 nm corresponding to the absorbance maximum of Doxorubicin was determined.

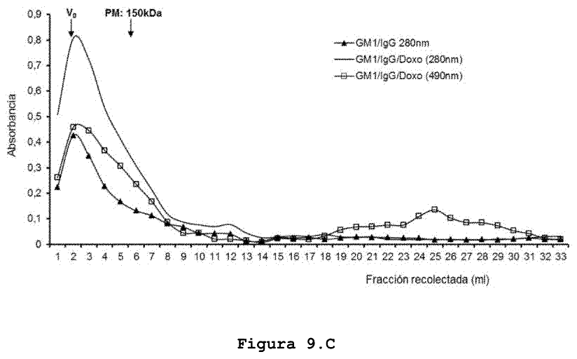

Doxorubicin elutes from fraction 22 (see FIG. 9. A); in contrast, in the presence of GM1, it elutes together with the GM1 micelle in the first fractions (1-4), which corresponds to the column exclusion volume (VO) determined with Dextran Blue. The GM1/Doxo complex exhibits similar absorbance maxima at 280 nm and 490 nm with a PM above 250 kDa.

The peak observed in the elution profiles of an IgG solution (5 mg/ml) at 280 nm in fractions 5 through 9 coincides with the 150 kDa PM corresponding to an immunoglobulin (see FIG. 9.B). Incubation of IgG with GM1 or GMI/Doxo micelles produces a shift in the IgG elution profile from a PM of 150 kDa to the fraction corresponding to VO (fractions 1-4) (see FIG. 9.C).

### Example 11: Effect of the Order of Incorporation of IgG and Doxorubicin in GM1 Micelles

Micelles of GM1 (10 mg/ml) in buffer pH 4.5 were heated at 55° C. for 1 hour. They were then allowed to stand until room temperature (20° C.) was reached and then IgG and Doxorubicin were incorporated according to the following procedure:

1.—A fixed amount of IgG (2.5 mg/ml) was added on top of GM1 micelles (10 mg/ml), and the mixture was incubated at pH 4.5, for 1 hour at 20° C.

2.—A fixed amount of Doxorubicin (0.7 mg/ml) was added on the GM1 micelles (10 mg/ml), and the mixture was incubated at pH 4.5, for 1 hour at 20° C.

3.—IgG (2.5 mg/ml) was added and incubated again for 1 hour at 20° C.

4.—Both samples, (A) those in which IgG was loaded first in the micelles and then Doxo; and (B) where Doxo was loaded first in the micelles and then IgG, were centrifuged at 15,000×g for 15 minutes and subjected to molecular filtration on Sephadex G 200 to separate the GM1/Doxo/IgG complexes from the free IgG. The absorbance spectra at 280 nm and 490 nm of the elution profile of IgG and Doxorubicin were determined corresponding to VO (fractions 1-4) (see FIG. 9.C).

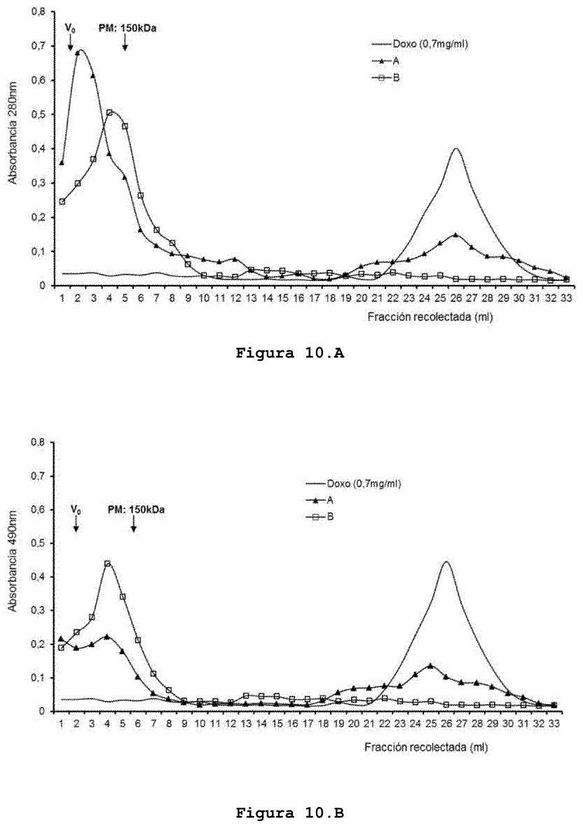

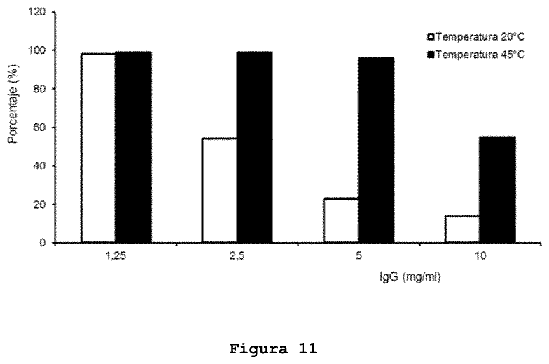

The amount of IgG that appears forming the GM1/Doxo/IgG complex is increased when it is incorporated according to procedure (A), at the same time that a fraction of free Doxorubicin is observed (see FIG. 10. A and 10. B). It was observed that when IgG is incorporated according to procedure (B) after incorporation of Doxorubicin, there is a shift of IgG to the free form (fractions 5-9).

### Example 12. Effect of Temperature on IgG Incorporation on Disialoganglioside (GD1) Micelles

GD1a and GD1a-Ptx micelles were prepared in acetic/acetate buffer pH 4.5, containing 20 mg/ml GM1, and a molar ratio of Ptx-GM1 1/20. Subsequently, they were incubated in the presence of increasing amounts of IgG (1.25-2.5-5 and 10 mg/ml). The incorporation of IgG on GDi-Ptx micelles was performed at pH 4.5 for 1 hour, at two different temperatures, 25° C. and 45° C. After incubation, samples were centrifuged at 15,000×g for 15 minutes and subjected to molecular filtration on Sephadex G 200 so as to separate GDi-Ptx-IgG complexes from free IgG. The IgG incorporated in the GDi micelle was determined by absorbance at 280 nm against an IgG standard. At higher temperature, the amount of IgG appearing forming the GDi-Ptx-IgG complex increased.

1. A pharmaceutical compound soluble in aqueous solution comprising:
at least a sialoglycosphingolipid; at least one polypeptide selected from the group consisting of antibodies, fragments and variants thereof; and at least one therapeutically active substance; wherein a ternary micellar complex if formed; and wherein said polypeptides are associated in a non-covalent manner with the micelles formed by said sialoglycosphingolipids and said therapeutically active substance.

2. The pharmaceutical composition of claim 1 characterized in that said sialoglycosphingolipids are selected from the group consisting of monosialogangliosides, disialogangliosides, trisialoganglioside, GM1, LIGA-GM1, GM2, GD1a, GD1b, GT1, and mixtures thereof.

3. The pharmaceutical composition of claim 1 characterized in that said polypeptides are selected from the group consisting of polyclonal human antibodies, polyclonal humanized antibodies, polyclonal antibodies of animal origin, monoclonal antibodies of animal origin, chimeric antibodies, fragments thereof, and mixtures thereof.

4. The pharmaceutical composition of claim 1 characterized in that said therapeutically active substance is selected from the group consisting of antitumor drugs, antifungal drugs, hydrophobic drugs, hydrophilic drugs, taxanes, paclitaxel, docetaxel, doxorubicin, amphotericin, prostaglandins, isosorbide dinitrite, prostaglandins, propofol, isosorbide dinitrate, testosterone, nitroglycerin, estradiol, vitamin E, cortisone, dexamethasone and its esters and betamethasone valerate in a molar ratio of therapeutically active substance/sialoglycosphingolipids in the range from 1:2 and 1:100.

5. The pharmaceutical composition of claim 1 characterized in that said therapeutically active substance is selected from the group consisting of paclitaxel, docetaxel, doxorubicin, and amphotericin.

6. The pharmaceutical composition of claim 1 characterized in that said therapeutically active substance comprises a molar ratio of therapeutically active substance/sialoglycosphingolipids in the range from 1:2 to 1:100.

7. The pharmaceutical composition of claim 1 characterized in that it comprises a sterile and translucent injectable composition, wherein said micelles have an average size of less than 100 nm.

8. The pharmaceutical composition of claim 1 characterized in that said micellar complex possesses an average size comprised between 10 nm and 60 nm.

9. The pharmaceutical composition of claim 1 characterized in that it directs the administration of the therapeutically active substance towards the target of said antibody.

10. The pharmaceutical composition of claim 1 characterized in that it comprises said micellar complex in the absence of albumin, linkers, crosslinking agents and derivatized molecules.

11. The pharmaceutical composition of claim 1 characterized in that said polypeptides of the ternary micellar complex retain the ability to bind to their ligand.

12. A pharmaceutical composition, soluble in aqueous solution comprising at least a sialoglycosphingolipid and a therapeutically active substance, which form micelles with the ability to non-covalently bind antibodies on their surfaces, to form a ternary micellar complex, Sialoglycosphingolipid-Therapeutically active substance-Antibody.

13. A process for obtaining the pharmaceutical composition of claim 1 comprising the following steps:
(a) solubilizing at least a sialoglycosphingolipid, in buffer solution or in a saline solution of pH 4.5, at a concentration higher than the critical micellar concentration, and heating at a temperature of between 45 and 60° C., for a time greater than 15 minutes and then allowing the solution to stand to form micelles;
(b) adding a solvent solution containing a therapeutically active substance;
(c) incubating the mixture of step (b) for incorporation of said therapeutically active substance into the micelles;
(d) dialyzing the micellar solution containing the therapeutically active substance of step (c) against a solution of distilled water or a pharmaceutically acceptable solution having a pH between 4 and 7, so as to remove the solvent;
(e) incubating the micelles resulting from step (d) in the presence of said polypeptide at a pH between 4 and 7.6, so as to allow the formation of the ternary complex Sialoglycosphingolipid-Therapeutically Active Substance-Polypeptide;
(f) sterilizing the aqueous and transparent solution obtained from the previous step.

14. The process of claim 13 characterized in that it further comprises the step of: (g) lyophilizing the sterilized micellar complex obtained in step (f).

15. The process of claim 13 characterized in that it further comprises the step of: (h) resuspending the lyophilisate of step (g) in a pharmaceutically acceptable solution at the time of use.

16. The process of claim 13 characterized in that said solvent is selected from the group consisting of a organic solvent, dimethyl sulfoxide, and ethanol.

17. The process of claim 13 characterized in that in step (f) the sterilization is carried out by filtration.

18. The process of claim 13 characterized in that in step (e) the mass ratio between sialoglycosphingolipids and polypeptide is between 2:1 and 6:1 (w/w), and said incubation is carried out for a time between 1 and 2 hours at a temperature of at least 45° C.

19. The process of claim 13 characterized in that said polypeptides are selected from the group comprising polyclonal human antibodies, polyclonal humanized antibodies, monoclonal humanized antibodies, polyionic antibodies of animal origin, monoclonal antibodies of animal origin, chimeric antibodies, fragments thereof and mixtures thereof.

20. The process of claim 13 characterized in that said sialoglycosphingolipids are selected from the group consisting of monosialogangliosides, disialogangliosides, trisialoganglioside, GM1, LIGA-GM1, GM2, GD1a, GD1b, GT1, and mixtures thereof.

21. The process of claim 13 characterized in that said therapeutically active substance is selected from the group consisting of antitumor drugs, antifungal drugs, hydrophobic drugs, hydrophilic drugs, taxanes, paclitaxel, docetaxel, doxorubicin, amphotericin, prostaglandins, isosorbide dinitrite, prostaglandins, propofol, isosorbide dinitrate, testosterone, nitroglycerin, estradiol, vitamin E, cortisone, dexamethasone and its esters, and betamethasone valerate in a molar ratio of therapeutically active substance/sialoglycosphingolipids ranging from 1:2 y 1:100.

22. The process of claim 13 characterized in that in step (e) the pH of the micelles is in the range between pH 4 and 7 in which said micelles are heated at a temperature between 45 and 65° C. for 1 hour.

23. The process of claim 13 characterized in that it comprises docetaxel as therapeutically active substance and as sialoglycosphingolipids: GM1, present in a docetaxel/GM1 molar ratio comprised between 1:10 and 1:100.

24. The process of claim 13 characterized in that it comprises as therapeutically active substance between 0.1 mg/ml to 6 mg/ml of paclitaxel or docetaxel and between 200 and 300 mg/ml of sialoglycosphingolipids.

25. The process of claim 13 characterized in that said therapeutically active substance comprises doxorubicin in a doxorubicin/sialoglycosphingolipid molar ratio comprised between 1:2.5 and 1:20.

26. A process for obtaining the pharmaceutical composition soluble in aqueous solution, of claim 1 characterized in that it comprises the following steps:
a—solubilizing sialoglycosphingolipids: GM1 in acetic-acetate buffer solution or in a saline solution of pH 4.5 always above the critical micellar concentration to obtain a sialoglycosphingolipid solution, and letting the first sialoglycosphingolipid solution stand for 24 hs;
b—heating the sialoglycosphingolipid solution at 60° C. for 1 hour, and then letting it cool down to room temperature;
c—adding an IgG solution at pH 4.5 to the sialoglycosphingolipid solution at room temperature and allowing it to stand for 2 hr;
d—adding a DMSO solution containing paclitaxel or docetaxel to the sialoglycosphingolipid solution;
e—dialyzing the sialoglycosphingolipid solution to remove the solvent and unincorporated drug;
f—sterilizing the sialoglycosphingolipid solution by filtration at 0.2 μm;
g—lyophilizing the sialoglycosphingolipid solution.

27. A pharmaceutical composition soluble in aqueous solution, characterized in that it is obtainable by the process of claim 13.

28. A pharmaceutical composition soluble in aqueous solution, characterized in that it is obtainable in step d) of the process of claim 13.
